(19) United States
(12) Patent Application Publication
(10) Pub. No.: US 2020/0407235 A1
(43) Pub. Date: Dec. 31, 2020

(54) METHOD FOR PREPARING A CAMGAL MIXED OXIDE, A CAMGAL MIXED OXIDE OBTAINABLE THIS WAY AND THE USE THEREOF FOR OLIGOMERIZATION OF GLYCEROL

(71) Applicant: SCG Chemicals Co., Ltd., Bangkok (TH)

(72) Inventors: Aunchana WANGRIYA, Bangkok (TH); Chawalit NGAMCHARUSSRIVICHAI, Bangkok (TH); Prissana SANGKHUM, Bangkok (TH)

(73) Assignee: SCG Chemicals Co., Ltd., Bangkok (TH)

(21) Appl. No.: 16/970,065

(22) PCT Filed: Jan. 7, 2019
(86) PCT No.: PCT/EP2019/050196

§ 371 Date: Aug. 14, 2020

(30) Foreign Application Priority Data
Feb. 15, 2018 (EP) 18156962.5

(51) Int. Cl. C01F 7/00; B01J 23/00; C07C 41/09

(52) U.S. Cl. CPC C01F 7/002; B01J 23/002; B01J 2523/23; B01J 2523/31; B01J 2523/22; C07C 41/09

(57) ABSTRACT

The present invention relates to a method for preparing a CaMgAl mixed oxide comprising the steps: a) providing a modified layered double hydroxide of the formula (I) wherein in formula (I) 0<x<0.9; b is from 0 to 10, preferably 1 to 10; c is from 0 to 10, preferably 1 to 10» and the AMO-solvent is an organic solvent miscible with water; b) calcining the modified layered double hydroxide; c) reacting the calcined modified layered double hydroxide with a calcium salt in the presence of an (a) organic acid; and d) calcining the product obtained in step c) to obtain the CaMgAl mixed oxide; a CaMgAl mixed oxide obtainable this way; and the use thereof.

The presented invention relates to a CaMgAl mixed oxide, the preparation thereof and a method for the production of polyglycerols using the same as a catalyst.

A variety of mixed oxides comprising Ca, Mg or Al or mixtures thereof are in the prior art. Likewise, the use of respective mixed oxides for catalyzing the production of oligo- and polyglycerols is described.

WO2010/044531 A1 discloses the production of linear polyglycerols via etherification of glycerol using (CaO)ₐ*(Ca₁₂Al₁₄O₃₃)₁₀₀₋ₐ, wherein “a” refers to a weight ratio of CaO based on 100 parts by weight of the total catalyst. The respective metal oxide catalysts were prepared from CaAl layered double hydroxides (LDHs) via co-precipitation followed by calcination. However, the method suffers from a variety of disadvantages, for example difficulties to control the metal composition and low diglycerols selectivity.

U.S. Pat. No. 5,721,305 A discloses the polymerization of glycerol in the presence of anionic clay catalysts. In this regard, the use of MgAl hydrotalcite, prepared by co-precipitation, is mentioned to be preferred. However, this method suffers from the disadvantages of high reaction temperature, low diglycerols selectivity and low glycerol conversion rates.

Perez-Barrado et al., Chemical Engineering Journal, 2015, 264, 547 discloses the etherification of glycerol catalyzed by MgAl LDH and CaAl LDH. According to this disclosure, the LDHs were prepared by co-precipitation followed by calcination at 450° C. However, the method suffers from low diglycerols selectivity and high acrolein selectivity.

Garcia-Sancho et al., Catalysis Today, 2011, 167, 84 is related to the use of MgAl LDH-derived mixed oxides, which are obtainable by co-precipitation or urea hydrolysis and the use thereof as base catalysts for glycerol etherification. However, this method suffers from low glycerol conversion.

Gholami et al., Journal of the Taiwan Institute of Chemical Engineers, 2013, 44, 117 discloses glycerol etherification catalyzed by Ca₁₊ₓAl₁₊ₓLaₓO₃ composites, which were synthesized via co-precipitation followed by calcination at 560° C. However, this method has several disadvantages, in particular requires high reaction temperature and shows low diglycerols selectivity.

It is, therefore, the object of the present invention to provide mixed oxide materials (or methods for preparing the same) overcoming drawbacks of the prior art, in particular mixed oxide materials, which may be used as catalysts in the glycerol oligomerization for improving diglycerol selectivity.

This object is first of all achieved by a method for preparing a CaMgAl mixed oxide comprising the steps: a) providing a modified layered double hydroxide of the formula (I)

[Mg₁₋ₓAlₓ(OH)₂]CO₃.b(H₂O).c (AMO-solvent) (I)

wherein in formula (I) 0<x<0.9; b is from 0 to 10, preferably 1 to 10; c is from o to 10, preferably 1 to 10, and the AMO-solvent is an organic solvent miscible with water; b) calcining the modified layered double hydroxide; c) reacting the calcined modified layered double hydroxide with a calcium salt in the presence of an organic acid; and d) calcining the product obtained in step c) to obtain the CaMgAl mixed oxide.

In this regard, it may be preferred that in the compound of formula (I) the ratio of Mg:Al is from 2 to 4.

The modified layered double hydroxide of the above formula (I) may be prepared by a method comprising precipitation of a layered double hydroxide and dispersing the same in the AMO-solvent (followed by a drying step). Further details as to the method of preparing the modified layered double hydroxide provided in the above step a) can be taken from the published patent applications WO 2014/051530 or WO 2015/144778.

In a preferred embodiment, the AMO-solvent is a solvent having a polarity (P) in the range from 3.8 to 9. Solvent polarity (P) is defined based on experimental solubility data reported by Snyder and Kirkland (Introduction to modern liquid Chromatography, 2^{nd} Edition; John Wiley and Sons, New York, 1979; pp 248-250. Particularly preferred solvents in this regard are acetone, acetonitrile, dimethylformamide, dimethyl sulphoxide, dioxane, ethanol, methanol, n-propanol, isopropanol or tetrathydrofuran. The most preferred solvents are methanol and acetone. Best results were achieved using acetone.

It is further preferred that the amount of calcium in the CaMgAl mixed oxide is 2-10 wt. %, preferably 5 to 8 wt. %, with respect to the weight of the calcined modified layered double hydroxide. By using the preferred amounts of calcium in the CaMgAl mixed oxide, best results as to diglycerols selectivity and diglycerols yield were achieved.

Furthermore, it is preferred that calcining in step b) is from 300 to 700° C., preferably for 1 to 10 h.

It is further preferred that the organic acid used in step c) is selected from citric acid, malic acid, tartaric acid, oxalic acid, succinic acid, formic acid, acetic acid.

Furthermore, it is preferred that reacting in step c) is an aqueous solution. Alternatively, other solvents, such as short chain alcohols, such as methanol and ethanol, suitable to dissolve the calcium salt may be used instead of water as a solvent in step c).

It is further preferred that reacting of step c) is in the further presence of a weak base, preferably ammonium hydroxide.

Ammonium hydroxide is ammonia water (herein 30 wt. % NH₃ in water). It is also possible to use organic amines, such as methylamine, ethylamine, to adjust the pH of citric acid solution. Methylamine or ethylamine in water, methanol and ethanol, at various concentrations, are available commercially. Alkaline hydroxides (strong bases), such as NaOH and KOH, are cheaper, and can be used for the pH adjustment, but the metal ions remain in the final mixed oxides and affect the catalysts basicity.

In a further embodiment reacting in step c) is at a pH from 5 to 7.

Furthermore, it is preferred that a step of drying the material obtained in step c) is carried out before performing step d)

It is further preferred that calcining in step d) is at a temperature from 450 to 900° C.

The object is further achieved by a CaMgAl mixed oxide obtainable by the inventive method.

Furthermore, the object is achieved by the use of the inventive CaMgAl mixed oxide in the oligomerization of glycerol.

Preferably, the oligomerization includes preparing diglycerols from glycerol.

Finally, the object is achieved by a method for oligomerization of glycerol comprising a step of contacting glycerol and the inventive CaMgAl mixed oxide.

In this regard, it is preferred that the amount of CaMgAl mixed oxide is from 2 to 10 wt.-%, preferably 2 to 5 wt. %, preferably 2.5 to 4.5 wt. %, even more preferred 2.5 to 3.5 wt.%, with respect to the weight of glycerol. In this way, best diglycerols selectivity was obtained.

Furthermore, it is preferred that contacting is carried out at a temperature from 150 to 300° C., preferably 200 to 230° C. In the above temperature ranges, best diglycerols selectivity and/or diglycerol conversion level was achieved.

It is therefore preferred that the contacting is for 1 to 48 h, preferably 4 to 40 h, even more preferred 8 to 32 h. In the preferred time ranges, best diglycerols selectivity and diglycerols yield were achieved.

It was surprisingly found by the inventors that the inventive method, in particular the key step c) thereof of reacting an AMO—Mg₃Al—0O₃ LDH material with a calcium salt in the presence of an organic acid results in a CaMgAl mixed oxide material having unique structural properties and, therefore, showing improved catalytic activity in the oligomerization of glycerol in particular is suitable to improve the diglycerols selectivity during oligomerization of glycerol.

In the following, the invention will be described in greater detail referring to the specific examples and Figures, without, however, the intention to respectively limit the scope of the invention.

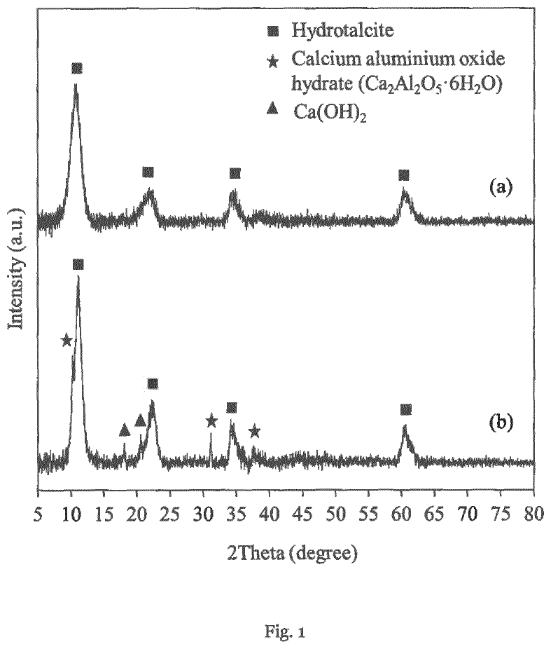

FIG. 1 XRD patterns of reconstructed 7.5% Ca—MgAl LDH (a) prepared by method according to the invention and (b) prepared by conventional impregnation.

### EXAMPLES

### Preparation of CaMgAl Mixed Oxide

### Comparative Example A

Modified layered double hydroxide of the formula (I) as described above (AMO-LDH) was prepared according to WO2014051530. A metal precursor solution was prepared by dissolving 1.575 mol (403.85 g) of Mg(NO₃)₂.6H₂O and 0.525 mol (196.94 g) of Al(NO₃)₃.9H₂O in 700 mL deionized water. The metal precursor was added dropwise into a 700 mL of Na₂CO₃ (0.315 mol, 33.39 g) solution. The pH of precipitation solution was adjusted to 10 using a NaOH solution in deionized water (4 M). The precipitant was aged at room temperature for about 3 h. LDH was collected by filtration, washing with deionized water until the filtrate pH was neutral, and rinsing with ethanol. The washed “wet cake” was dispersed in ethanol. After stirring for about 1 h, the sample was filtered. The final AMO-LDH product was dried overnight in the fume hood at room temperature and then in an oven at 110° C. for 12 h.

### Example 1

AMO-LDH, prepared in the same manner as Comparative example A, was calcined, to obtain mixed oxide of Mg and Al, in a muffle furnace at 450-550° C. for 1-5 h. Citric acid solution was prepared by dissolving 2.67 g of citric acid in 2.67 g of deionized water, and adding ammonium hydroxide (30 wt. % ammonia in water) to adjust the solution pH to 5-7. Then, 8.84 g of Ca(NO₃)₂.4H₂O were dissolved in the citric acid solution, followed by slowly dropping the Ca solution onto 20 g of the calcined AMO-LDH under stirring. The wet powder was dried in an oven at 100° C. overnight to obtain reconstructed CaMgAl LDH. The sample was analyzed using X-ray diffraction method. Then, it was calcined in a muffle furnace at 650-800° C. for 5 h. The CaMgAl mixed oxide (CaMgAl LDO) with 7.5 wt. % of Ca content was obtained.

FIG. 1(a) shows XRD pattern of reconstructed CaMgAl LDH with 7.5 wt. % of Ca content. It consists of pure hydrotalcite phase. There was no separated Ca phase and impurities.

### Example 2

Example 2 was prepared in the same manner as Example 1, except 5.89 g of Ca(NO3)₂.4H₂O was used to obtain 5 wt. % Ca content.

### Conventional impregnation (Comparative Example B)

AMO-LDH was prepared and calcined following the method of the invention above. Then, it was impregnated with Ca(NO₃)₂.4H₂O in water by slowly dropping 8.84 g of Ca(NO₃)₂.4H₂O in water on the calcined AMO-LDH under stirring. The wet powder was dried in an oven at 100° C. overnight to obtain the reconstructed CaMgAl LDH. Then, it was calcined in a muffle furnace at 650-800° C. for 5 h. The CaMgAl mixed oxide with 7.5 wt % of Ca content was obtained.

FIG. 1(b) shows the reconstructed CaMgAl LDH prepared by conventional impregnation. It consists of Ca(OH)₂ and CaCO₃ as impurity phases.

### Production of Short-Chain Polyglycerols

50 g of glycerol were added into a three-necked round-bottom flask equipped with a N₂ gas line and Dean-Stark apparatus to continuously remove water generated as a by-product. The glycerol heated to the reaction temperature under stirring using a heating mantle. Then, the CaMgAl catalyst was added into the flask. The reaction was carried out with 3 wt. % of catalyst loading level with respect to the weight glycerol used at temperature of 220° C. for 24 h.

The reaction product was diluted with 100 mL of methanol to reduce product viscosity, and then filtered to separate the catalyst. The methanol was removed by rotary evaporator. product was analyzed for the glycerol conversion and the products yield using a gas chromatography. The glycerol conversion, diglycerols yield and diglycerols selectivity were calculated by the following equations.

$$Glycerol conversion ( mol \% ) = \frac{Mole of converted glycerol}{Mole of initial glycerol} \times 100$$

$$Diglycerols selectivity ( \% ) = \frac{Mole of diglycerols}{Mole of converted glycerol} \times 100$$

Comparative examples A shows 36% glycerol conversion and 51% diglycerols selectivity, while comparative example B shows 55% glycerol conversion and 50% diglycerols selectivity. The inventive examples 1 and 2 show higher diglycerols selectivity of 78% and 66%, respectively because it consists pure phase of CaMgAl LDO resulting in high dispersion of Ca atoms. The interaction between Ca, Mg and Al oxides formed a synergetic mixed oxide complex, which provided an enhanced basicity and contributed to the high catalytic performance during glycerol etherification. While, the conventional CaMgAl LDO consists of less basic oxides, and gives low diglycerols selectivity.

The features disclosed in the foregoing description and in the claims may, both separately and in any combination be material for realizing the invention in diverse forms thereof.

1. A method for preparing a CaMgAl mixed oxide comprising the steps:
a) providing a modified layered double hydroxide of the formula (I)
[Mg₁₋ₓAlₓ(OH)₂]CO₃.b(H₂O).c (AMO-solvent) (I)
wherein in formula (I) 0<x<0.9; b is from 0 to 10; c is from 0 to 10, and the AMO-solvent is an organic solvent miscible with water;
b) calcining the modified layered double hydroxide;
c) reacting the calcined modified layered double hydroxide with a calcium salt in the presence of an organic acid; and
d) calcining the product obtained in step c) to obtain the CaMgAl mixed oxide.

2. The method according to claim 1, wherein the amount of the calcium salt is 5-10 wt. %, with respect to the weight of the calcined modified layered double hydroxide.

3. The method according to claim 1, wherein calcining in step b) is from 300 to 700° C., for 1 to 10 h.

4. The method according to claim 1, wherein reacting of step c) is in the further presence of a weak base.

5. The method according to claim 1, wherein reacting in step c) is at a pH from 5 to 7.

6. The method according to claim 1, further comprising a step of drying the material obtained in step c) before performing step d).

7. The method according to claim 1, wherein calcining in step d) is at a temperature from 450 to 900° C.

8. The method according to claim 1, wherein the organic acid is selected from the group consisting of citric acid, malic acid, tartaric acid, oxalic acid, succinic acid, formic acid, and acetic acid.

9. The method according to claim 1, wherein reacting in step c) is an aqueous solution.

10. A CaMgAl mixed oxide produced by the method according to claim 1.

11-12. (canceled)

13. A method for oligomerization of glycerol comprising a step of contacting the glycerol and the CaMgAl mixed oxide according to claim 10.

14. The method according to claim 13, wherein the amount of CaMgAl mixed oxide is from 2 to 10 wt. % with respect to the weight of glycerol.

15. The method according to claim 13, wherein contacting is carried out at a temperature from 150 to 300° C.

16. The method according to claim 1, wherein, in formula (I), b is from 1 to 10.

17. The method according to claim 1, wherein, in formula (I), c is from 1 to 10.

18. The method according to claim 2, wherein the amount of the calcium salt is 7-8 wt. %, with respect to the weight of the calcined modified layered double hydroxide.

19. The method according to claim 4, wherein the weak base is ammonium hydroxide.

20. The method according to claim 15, wherein the temperature is from 200 to 230° C.
